(19) United States
(12) Patent Application Publication
(10) Pub. No.: US 2018/0003705 A1
(43) Pub. Date: Jan. 4, 2018

(54) BIOSENSOR FOR USE WITH A SURFACE PLASMON RESONANCE (SPR) SENSOR

(71) Applicant: Research Foundation of the City University of New York, New York, NY (US)

(72) Inventors: Ronald Koder, New York, NY (US); Peter Schnatz, New York, NY (US); Joseph Brisendine, New York, NY (US); David Crouse, Potsdam, NY (US); Lori Lepak, Potsdam, NY (US); Igor Bendoym, Potsdam, NY (US)

(21) Appl. No.: 15/595,458

(22) Filed: May 15, 2017

Related U.S. Application Data
(60) Provisional application No. 62/336,197, filed on May 13, 2016.

(51) Int. Cl. G01N 33/543; G01N 21/552; G01N 33/552; G01N 33/553

(52) U.S. Cl. CPC G01N 33/54373; G01N 33/553; G01N 21/554; G01N 33/552

(57) ABSTRACT

A surface plasmon resonance (SPR) sensor is provided that has enhanced sensitivity. The sensor's plasmonic chip has intrinsically disordered proteins (IDPs) that undergo enzyme-free folding upon binding to an analyte. This binding results in a detectable change in refractive index and thereby permits detection of the analyte.

### CROSS-REFERENCE TO RELATED APPLICATIONS

This application claims priority to and is a non-provisional of U.S. Patent Application 62/336,197 (filed May 13, 2016), the entirety of which is incorporated herein by reference.

### STATEMENT OF FEDERALLY SPONSORED RESEARCH OR DEVELOPMENT

This invention was made with government support under grant number FA4819-14-C-0017 awarded by Air Force Civil Engineer Center and grant number ECCS-1542081 awarded by the National Science Foundation. The government has certain rights in the invention.

### REFERENCE TO A SEQUENCE LISTING

This application refers to a “Sequence Listing” listed below, which is provided as an electronic document submitted herewith which is incorporated herein by reference in its entirety.

### BACKGROUND OF THE INVENTION

The subject matter disclosed herein relates to optical biosensor and specifically to surface plasmon resonance (SPR) biosensors. Advances in the field of optical biosensors have shown promise in diverse applications such as medical diagnostics, food safety and security. Label-free optical biosensors often measure refractive index changes caused by the binding of a target analyte to a surface. SPR biosensors use metal-dielectric surfaces as waveguides and represent one specific type of optical biosensor. These SPR biosensors have shown significant potential in this emerging field. It would therefore be desirable to provide an improved SPR biosensor for detecting a target analyte.

The discussion above is merely provided for general background information and is not intended to be used as an aid in determining the scope of the claimed subject matter.

### BRIEF DESCRIPTION OF THE INVENTION

A surface plasmon resonance (SPR) sensor is provided that has enhanced sensitivity. The sensor's plasmonic chip has intrinsically disordered proteins (IDPs) that undergo enzyme-free folding upon binding to an analyte. This binding results in a detectable change in refractive index and thereby permits detection of the analyte.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A is a schematic diagram showing intrinsically disordered proteins (IDPs) in an extended state;

FIG. 1B is a schematic diagram showing IDPs in a folded state;

FIG. 2A is a schematic cross section view of a device useful as a surface plasmon resonance (SPR) sensor;

FIG. 2B is a top view of a tray for use in the SPR sensor;

FIG. 3 is a graph showing a reflectance spectrum of reflectance as a function of incident angle;

FIG. 4 is a top view of a grid showing placement of IDPs on a flat substrate;

FIG. 5 is a cross section side view of a dielectric substrate showing a metasurface structure;

FIG. 6 is a graph that illustrates the change in transmission before and after the IDPs undergo folding;

FIG. 7A is a cross section view showing the IDPs in an elongated state while FIG. 7B is a IDPs in a folded state after the binding event;

FIG. 8 depicts a graph of transmission of grating as a function of wavelength before and after binding;

FIG. 9 is a flow diagram showing a method of forming a substrate;

FIG. 10A and FIG. 10B are scanning electron microscopy (SEM) of a gating structure viewed both directly from above (FIG. 10A) and in cleaved cross-section (FIG. 10B);

FIG. 11 is a graph showing absorption frequency shift upon heme binding to a model protein H4(−28); and

FIG. 12 is a schematic diagram showing two IDPs for a supercharged ricin-binding short-chain antibody fragment in an extended state and folded state.

### DETAILED DESCRIPTION OF THE INVENTION

This disclosure provides a highly sensitive handheld sensor device that uses intrinsically disordered proteins (IDPs) that are designed to undergo extreme conformational changes upon binding their target. These conformational changes cause extreme changes in refractive index in the protein layer. The proteins are attached to a detector chip with a structured metasurface to translate the refractive index change into an enhanced shift in surface plasmon resonances (SPR). This configuration significantly improves the sensitivity of the overall detector relatively to current commercially available SPR systems. Calculations demonstrate the conformational changes in the engineered proteins provides the desired change in refractive index. The device holds considerable promise as a low-cost, highly sensitive, field-deployable detection system for chemical and biological toxins.

A method is also provided that takes advantage of a protein that undergoes a dramatic conformational change upon binding to its target. This conformational changes is facilitated by coating the outside of the proteins with a very large number of like charges (e.g. a very large number of negative charges).

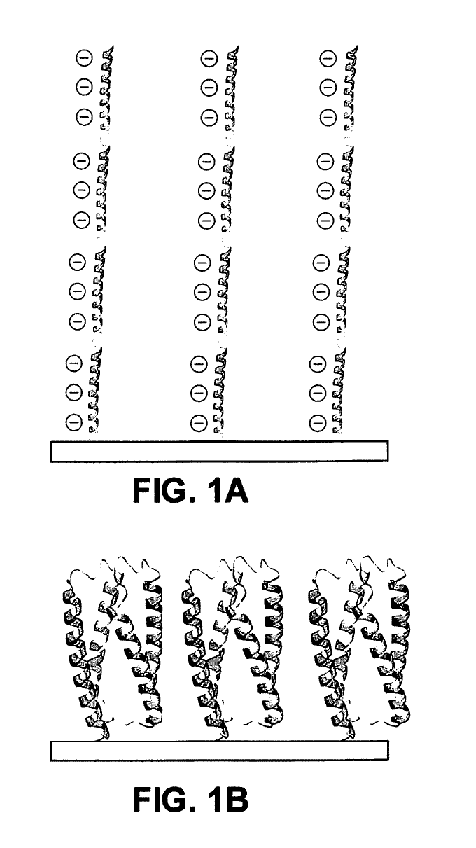

In disclosed method a ligand-binding protein and the environment the outside of the protein are engineered to have a large excess of negatively charged amino acid side chains such that the proteins have the property of binding induced folding, in which the protein goes from a ‘rigid rod’-like conformation to a folded natural protein upon ligand binding (see FIG. 1A and FIG. 1B). FIG. 1A depicts an extended ligand conformation attached to solid support (e.g. a gold film). FIG. 1B depicts folded ligands that formed upon binding to an analyte.

Intrinsically Disordered Proteins (IDPs) undergo a phase transition between a disordered state and a folded state as part of their function. While the majority of IDPs make this phase change in response to kinase or phosphatase action, a subclass of these proteins are unstructured until they bind to another protein or small molecule ligand—a process termed Ligand-Induced Folding. These proteins typically have a very high (e.g. more than 15%) net charge per residue, and commonly the net charge is a result of a very high (e.g. 3:1 negative:positive) charge ratio—for example, if the protein is negatively charged, there will be very few (e.g. less than 5%) if any positively charged side chains. This partially destabilizes the folded state of the protein by charge-charge repulsion and the additional folding energy that results from ligand binding is sufficient to drive the phase transition from an extended unliganded state to a folded bound state. In one embodiment, the IDPs are between two-hundred and three-hundred residues. This disclosure outlines the design of proteins which have this property, and the large electric field change that accompanies the folding of these proteins is ideal for high-sensitivity sensing. As would be recognized by those skilled in the art, negatively charged amino acids include aspartic acid and glutamic acid. Positively charged amino acids include arginine, histidine and lysine. As used in this specification, the charge refers to the charge the amino acid would adopt at physiological pH. IDPs typically include a combination of high net charge and low hydrophobic character. The interplay between these sequence specific characteristics will determine if a structure is natively unfolded. Using a normalized version of the Kyte and Doolittle approximation of amino acid hydrophobicity, the mean hydrophobicity per residue, [symbol] H [symbol] , can be calculated. In order for a protein domain to be intrinsically disordered, [symbol] H [symbol] and the absolute mean net charge per residue [symbol] R [symbol] must satisfy the following constraint:

2.785 [symbol] H [symbol] − [symbol] R [symbol] <1.151

Examples of IDPs include: the kinase-inducible domain (pKID) of the cyclic-AMP-response-element-binding protein (CREB) is unstructured in solution, but undergoes a folding transition upon binding to the CREB-binding protein (CBP). The free eukaryotic translation-initiation factor (eIF4E) has local disorder at its N terminus, but is structure when it binds with initiation factor 4G. The hypoxia-inducible factor-la (HIF1α), which regulates the hypoxic response, contains an unstructured C terminus when free in solution and has a folding transition when binding to the transcriptional-adaptor zing-finger-1 (TAZ1) domain of the transcriptional co-activators CBP/p300. The nuclear-receptor co-activator-binding domain (NCBD) of human CBP is molten globule when free of its full unstructured binding partner, the activator for thyroid hormone and retinoid receptors (ACTR); however they undergo synergistic folding and binding when forming a complex.

This method increases signal-to-noise 1,000-10,000-fold in the plasmonic sensing methods that are regularly used in medical testing and in the pharmaceutical industry. This both massively improves the sensitivity of existing laboratory tests and enables the creation of portable biosensors for the detection of biological weapons, drugs, poisons, and even hand-held versions of medical tests for community medicine.

IDPs have been found to be essential in cell biology, playing important roles in signal transduction and regulatory functions. Their functional dependence on a native unstructured state at physiological conditions has catalyzed a rapid increase in studies regarding IDP behavior. It has been shown that this class of proteins is completely unstructured or contains unstructured regions until an environmental signal or binding event occurs. The disclosed device and method uses a rational design of IDPs which exhibit coupled folding and binding behavior. The resulting large conformational change upon binding to a target analyte causes enhanced local changes in the refractive index.

Emergent computational techniques were used to identify or design natural or artificial IDPs that undergo a significant phase transition from a predominantly rigid coil to an ordered, folded structure upon analyte binding. Past studies on the amino acid sequences of IDPs have reported that a large net charge and reduced hydrophobicity can impart instability and result in a natively unfolded state at neutral pH. The disclosed device mimics these effects and focuses on incorporating high net charge by mutating the solvent exposed side chains of neutral proteins to basic (negative) residues. The electrostatic repulsion of the negative charges destabilizes the structure and forces it to take on characteristics similar to a random coil conformation. By increasing the solution ionic strength it is possible to screen the side chain charges and reduce the repulsive forces, switching the energetically favorable conformation from random coil to natively folded. Similarly, the addition of analyte will induce folding at an experimentally determined ionic strength, exhibiting the desired IDP behavior.

The disclosed device and method achieves several significant improvements over existing SPR biosensing instruments, including: (1) Increased Sensitivity: The system employs a new class of designed proteins with extraordinarily large conformational changes upon analyte binding, producing an orders-of-magnitude increase in sensitivity. (2) Robustness and compactness: All the optical components of the system are in line with each other, making for an easier, more robust, and more stable alignment than existing reflection-based systems. (3) Low-cost, multi-functional: The biosensing chip is planar (i.e., flat), inexpensive, disposable, and quickly interchangeable, allowing for multiple and repeated testing for different toxins in the field (e.g., battlefield, ports-of-entry, first responders, etc.)

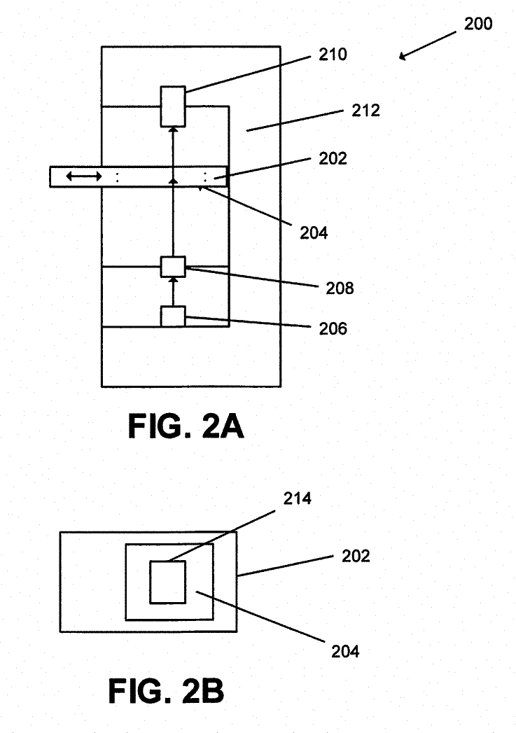

The device operates by detecting changes in the transmitted light (rather than reflected light) that occur with the binding of a particular target (e.g. a toxin) to the functionalized surface. An exemplary housing of the device is shown in FIG. 2A and FIG. 2B. FIG. 2A shows a bisected view of a complete housing 200 including a slide-in tray 202 that holds the plasmonic chip 214 and sample to be tested. In the embodiment depicted in FIG. 2A the sample wafer sits on a tray 202 with an exposed underside 204 to allow light to pass through the sample wafer. A narrow-bandwidth light source 206 illuminates the plasmonic chip 214 from below, exciting plasmon modes on the chip 214. Any binding event of a biotoxin alters the transmission through the chip 214 as measured by a detector 210 (e.g. a photodiode array) above. This configuration allows all the optical components to be in line with each other, making for a more-compact and more-robust system. These innovations form the basis of a transmission-based device with an easily replicable disposable sensing chip. From bottom to top, the components are a narrow-bandwidth light source 206 (e.g. a 850-nm, 10-mW laser diode), a lens 208 (e.g. a 4.51-mm aspheric collimating lens), a tray 202 (which may include a linear polarizer) and a photodiode array 210 (e.g. a 5.1-mm2-active-area silicon photodetector). The space 212 along the top, bottom, and side are intended to house batteries and circuitry. FIG. 2B is a top view of the tray 202.

The disclosed device utilizes surface plasmonic effects to create more-sensitive detection capabilities in the proposed transmission-based apparatus. The device incorporates a disposable, nanofabricated plasmonic chip (e.g. a metal-glass-semiconductor composite) in which the components are arranged in a metasurface structure—a highly organized, and periodically repeating pattern, with feature sizes on the scale of several hundred nanometers. In general, metasurface patterns can be simple grids, arrays of holes in a metal film, or more complex designs such as concentric circles or ellipses. By using a combination of custom software and commercial photonics packages to model the optical properties of these materials, the shapes of the patterns and the chemical composition of the component materials may be chosen to achieve the desired light-controlling behavior. For simplicity, nanopatterned grating structures with high depth-to-width ratios were chosen to transmit narrow wavelength ranges of light. These transmission bands shift in response to analyte binding of the detector proteins, as the engineered extreme change in protein conformation induces an extreme change in the index of refraction at the chip surface. The metasurface structure is designed to induce a detectable shift (ideally greater than 20 nm) in the transmission peak.

Preliminary investigations initially assumed the optics are in the Kretschmann configuration although, as discussed elsewhere in this specification, these results ultimately prove to be independent of geometry allowing other optical configurations, including transmission mode. The Kretschmann arrangement contains a gold film at varying angles. If the angle satisfies the dispersion relation for the three-layer system, the light will be absorbed and excite surface plasmons in the metal. Consequently, the reflectance will be reduced at that resonance angle. The reflectance can be computed as a function of incident angle by repeated application of the Fresnel equation. A gold film of thickness d_{g} has the following ray transfer matrix:

$$M = [ \begin{matrix}cos ( {k}_{yg} {d}_{g} ) & - \frac{i {ε}_{g}}{{k}_{yg}} sin ( {k}_{yg} {d}_{g} ) \\ - \frac{i {k}_{yg}}{{ε}_{g}} sin ( {k}_{yg} {d}_{g} ) & cos ( {k}_{yg} {d}_{g} )\end{matrix} ] .$$

Here, the y-component of the wave vector, k_{yg}, passing through the gold film can be determined from the incident angle, θᵢ, and the incident wavelength, λ, at the gold-prism interface

$${k}_{yg}^{2} = {{n}_{p}^{2} ( \frac{2 π}{λ} )}^{2} ( \frac{{n}_{g}^{2}}{{n}_{p}^{2}} - {sin}^{2} {θ}_{i} ) ,$$

where n_{g} and nₚ are the refractive indices of the gold and prism, respectively. The reflection coefficient, rₚ(θᵢ), is given by

$${r}_{p} ( {θ}_{i} ) = \frac{( {M}_{11} + {M}_{12} \frac{{k}_{ys}}{{ε}_{s}} ) \frac{{k}_{yp}}{{ε}_{p}} - ( {M}_{21} + {M}_{22} \frac{{k}_{ys}}{{ε}_{s}} )}{( {M}_{11} + {M}_{12} \frac{{k}_{ys}}{{ε}_{s}} ) \frac{{k}_{yp}}{{ε}_{p}} - ( {M}_{21} + {M}_{22} \frac{{k}_{ys}}{{ε}_{s}} )} .$$

The wave vectors in the prism and the sample are denoted k_{yp} and k_{ys}, respectively. Like k_{yg}, they are defined by the incident angle and the refractive indices of the configuration components:

$${k}_{yg}^{2} = {{n}_{p}^{2} ( \frac{2 π}{λ} )}^{2} {cos}^{2} {θ}_{i}$$

$${k}_{ys}^{2} = {{n}_{p}^{2} ( \frac{2 π}{λ} )}^{2} ( \frac{{n}_{s}^{2}}{{n}_{p}^{2}} - {sin}^{2} {θ}_{i} ) ,$$

where nₛ is the refractive index of the sample just above the gold film in the flow chamber (FIG. 2A). Finally, the reflectance as a function of incident angle is given by

R(θᵢ)=|r(θ₁)|²

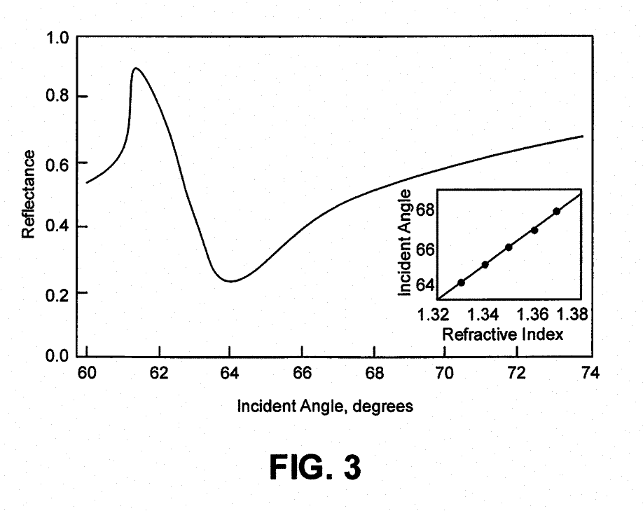

Because the dielectric constant and the refractive index are intimately related (n=\sqrt{∈}) any change to the refractive index at the surface of the gold film in the sample chamber will cause a shift in the angle of minimum reflectance. A typical reflectance spectrum for the parameters in Table 1 is depicted in FIG. 3.

TABLE 1
Parameters for reflectance spectrum shown in FIG. 3.

| Parameter | Value |
|---|---|
| nₚ | 1.517 |
| εₚ | 2.301 |
| n_{g} | 0.19591 |
| ε_{g} | −10.575 + i1.2765 |
| nₛ | 1.33 |
| εₛ | 1.7689 |
| dₘ | 60 nm |
| λ | 633 nm |

Here the minimum angle of reflectance is 64.06°; this angle varies linearly with the refractive index of the sample nₛ (FIG. 3, insert). The slope of a fitted line to this plot is 95.35 indicating that, for an increase in refractive index of 0.1, there is a minimum reflectance angle shift of 9.535°. Furthermore, due to the relative refractive indices of the three mediums, the wave passing into the sample chamber is, in fact, an evanescent wave with penetration depth 1/k_{ys}.

The presence of supercharged IDPs on the surface of the gold will induce a binding-dependent refractive index at the gold-sample interface. After binding to the target analyte, the protein will undergo a large conformational change that will affect the refractive index of the sample just above the gold surface. To determine the magnitude of this change, the refractive index of the protein may be estimated using a method outlined by McMeekin et al (McMeekin, T. K., Groves, M. L., Hipp, N. J., “Refractive indices of amino acids, proteins, and related substances,” Amino Acids and Serum Proteins. American Chemical Society. Chapter 4, 54-66 (1964); McMeekin, T. L., Wilensky, M., Groves, M. L., “Refractive indices of proteins in relation to amino acid composition and specific volume,” Biochemical and Biophysical Research Communications, 7(2) 151-156 (1962). The refraction per gram of protein, Rₚ, is calculated as the mass-weighted average of the refraction per gram of the contributing amino acids Rₐ,

$${R}_{p} = \frac{{\sum }_{a} {R}_{a} {M}_{a}}{{\sum }_{a} {M}_{a}} ,$$

where Mₐ is the molecular mass of residue a. Additionally, the partial specific volume of the protein, vₚ, is calculated as the mass weighted partial specific volume, vₐ, of the contributing amino acids,

$${v}_{p} = \frac{{\sum }_{a} {v}_{a} {M}_{a}}{{\sum }_{a} {M}_{a}} .$$

Finally, by applying the Lorentz-Lorenz formula, an estimate of the refractive index of the protein is

$$n = \sqrt{\frac{2 {R}_{p} + {v}_{p}}{{v}_{p} - {R}_{p}}} .$$

Using the molar refraction of each amino acid reported by McMeekin, and the corresponding partial specific volume reported by Cohn (Cohn, E. J., Edsall, J. T., “Density and apparent specific volume of proteins,” Proteins, Amino Acids and Peptides, Van Nostrand-Reinhold, 370-381 (1943)), the typical refractive index for our IDP is nₚᵣₒₜ=1.514.

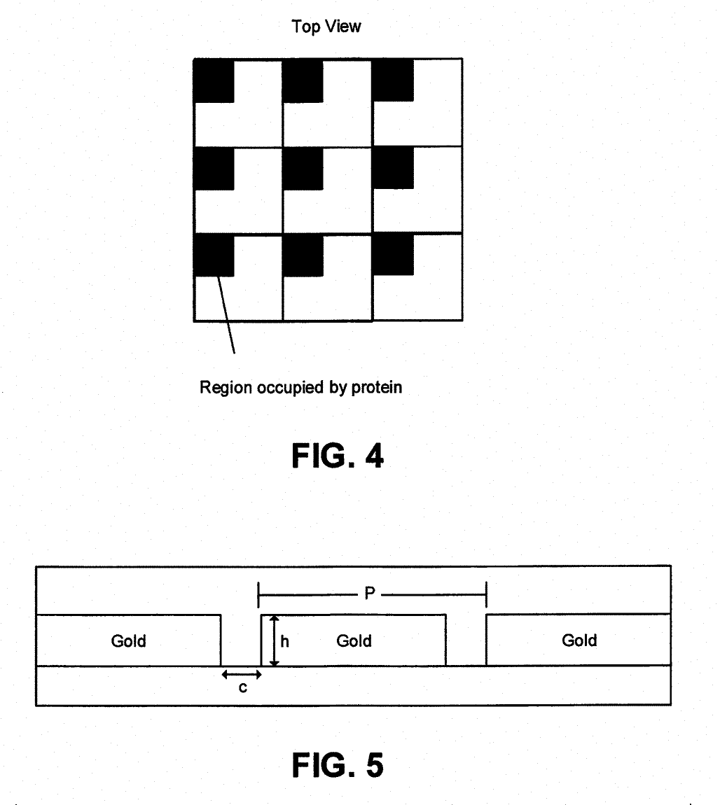

Due to the electrostatic repulsive forces discussed earlier, the protein can be assumed to have a rigid rod conformation when attached to the gold (FIG. 1A). The volume of the protein was approximated as a rectangular box with a square footprint of side length 1.25 nm and a height of 20.0 nm. For simplicity, each structure was assumed to sit on a grid, each occupying one corner of a 2.5-×2.5-nm square (FIG. 4). The interstitial space will be filled with water. A simple estimate of the total effective refractive index of the entire sample chamber, n_{eff,1}, has been established by Jung et al. (Jung, L. S., Campbell, C. T., Chinowsky, T. M., Mar, M. N., Sinclair, S. Y., “Quantitative Interpretation of the Response of Surface Plasmon Resonance Sensors to Adsorbed Films,” Langmuir, 15(19), 5636-5648 (1998)) for the Kretschmann geometry. The SPR signal is the result of the interaction of the evanescent field with the refractive index of the sample. For λ=633 nm, the penetration depth of this field is δ≈100 nm and the intensity decays as e^{-2y/δ}. The effective refractive index is determined by weighting the local refractive index by the intensity along the perpendicular distance from the gold surface. For this example, the first 20 nm have the volume-weighted refractive index of the water and protein, and farther out has the refractive index of water alone,

$$\begin{matrix}{n}_{eff , 1} = \frac{2}{δ} \{ {\int }_{0}^{20} ( 0.25 {n}_{prot} + 0.75 {n}_{w} ) {e}^{- 2 y / δ} dy + {\int }_{20}^{∞} {n}_{w} {e}^{- 2 y / δ} d \} \\ = 1.3553\end{matrix}$$

Here, n_{w}=1.33, the refractive index of water.

Once the target analyte is introduced, the protein will fold into a rigid structure at the surface of the gold, occupying the entire area of the 2.5-×2.5-nm square with a height of 5 nm (FIG. 1B). Similarly, the effective refractive index can be calculated,

$$\begin{matrix}{n}_{eff , 2} = \frac{2}{δ} \{ {\int }_{0}^{5} {n}_{prot} {e}^{- 2 y / δ} dy + {\int }_{5}^{20} {n}_{w} {e}^{- 2 y / δ} d y \} \\ = 1.3634 .\end{matrix}$$

This substantial increase in the effective refractive index upon ligand binding will result in a 0.77° increase of the angle of minimum reflectance, almost eighty times the typical SPR signal obtained for antibody/antigen interaction studies.

This calculation did not account for the charged residues on the IDP, and should be considered a lower limit to the effect of the conformational change on the refractive index. The carboxylate groups of the charged protein will contribute significantly to the polarizability of the structure, increasing the effective refractive index above the gold film. Additionally, strong image charges will be induced on the gold film, and may act as a strong perturbance to the resonance frequency of surface plasmon.

Although the analysis began by assuming that the device was configured in the Kretschmann geometry and operated in reflection mode, in the final analysis, the equations describing the change in refractive index caused by the change in protein conformation are not explicitly dependent upon the overall geometry of the device. Thus, other geometric configurations could be considered, including transmission-based systems. A transmission-based device has a simpler, easier-to-align, and more robust optical path than a reflection-based system. Thus, for one implementation of the device plasmonic chips, which are capable of operating in transmission mode, were chosen.

Computationally, the easiest metasurface structure to employ in simulations of optical properties is a periodic array of rectangular wires on a dielectric substrate, as shown in FIG. 5. The substrate is a fused silica wafer (n=1.48), which is transparent in the visible, to allow the device to operate in transmission mode. The wire grating is made of gold, in anticipation of using standard thiol chemistry to attach the engineered proteins directly to the metasurface, to maximize the enhancement in the SPR shift. The metasurface is fully immersed in a superstrate, which also fills the grooves between the gold wires, and is assumed to have the same refractive index as water. In FIG. 5 plasmonic structure were modeled as a grating which is periodic in one dimension. Rectangular metallic gold wires extend into and out of the cross-sectional perspective. The superstrate is a dielectric fluid that fills the grooves between gold wires, and the substrate is a dielectric material.

Preliminary simulations were performed using custom software. To maximize the shift in the SPR resonance upon binding, three geometric parameters of the structure were allowed to vary: the gold wire thickness (h); the wire-to-wire period (i.e., the period or pitch of the grating) (P); and the gap between gold wires (c). These simulations indicated that the optimal change in SP transmission in response to the binding of target molecules occurs when the period of the grating P=630 nm, the height h=50 nm, and the groove width is 420 nm.

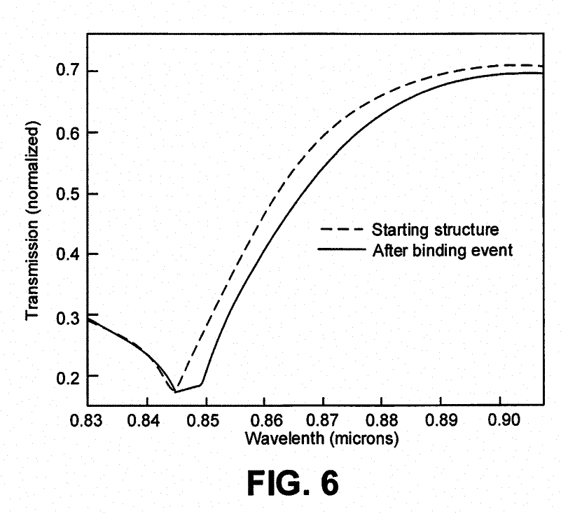

To model a binding event, a 20-nm thick layer atop the gold film within the superstrate, which was initially assumed to be pure water (n=1.34), experiences an increase in the index of refraction (to n=1.38), as the protein folds into a denser conformation near the gold surface. The changes are similar to what is expected even with the binding of very small molecular targets, such as heme. FIG. 6 illustrates the change in transmission for a grating with the optimized dimensions described above, before and after a small fraction of the attached proteins undergo the model binding event. The predicted change is of a magnitude which should be easily measurable by standard, commercially available silicon photodetectors. Furthermore, a plasmonic structure capable of detecting even this small index change, should be capable of detecting larger target molecules, such as proteins or even viral or bacterial pathogens, which should induce a larger refractive index change upon binding.

More-detailed simulations of the optical transmission through the gratings before and after binding were performed using COMSOL Multiphysics RF Module in the frequency domain. Given the geometry of the grating design, a two-dimensional study was performed, which significantly reduced computation time. The left and right walls were assigned periodic Floquet boundary conditions (i.e., periodic boundary conditions). The top and bottom walls were assigned as periodic ports. With both in-plane and out-of-plane diffractive orders calculated by COMSOL, there were five ports for the top and five for the bottom. As the actual implementation of this system uses polarized light, only with p-polarized light (TM polarization) incident from the bottom port was of concern. The period of the structure is 630 nm. The height of the upper and lower boxes (with index of refraction of 1.34) are 1.5 times the incident wavelength (about 880 nm) in the particular medium to ensure we are away from near-field effects when measuring S parameters.

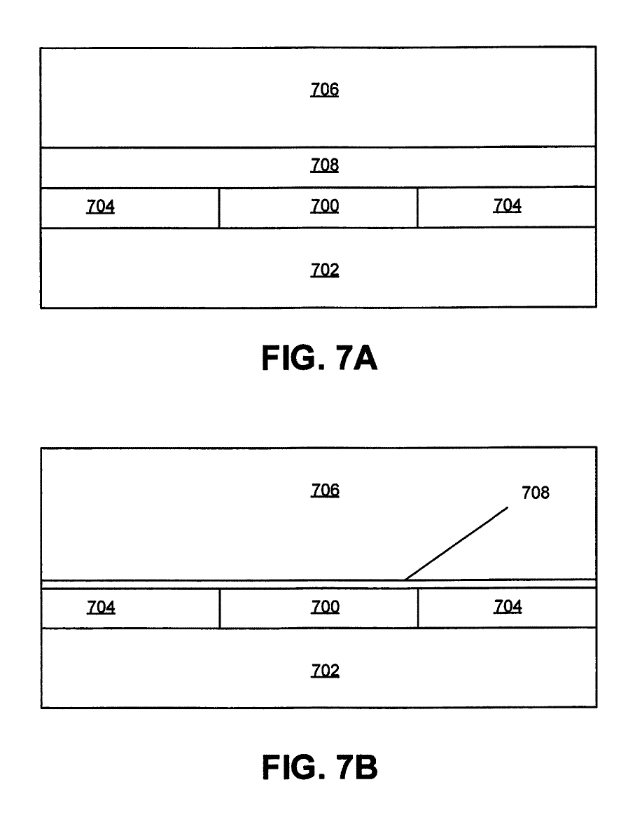

The grating structures that were simulated in COMSOL are shown in greater detail in FIG. 7A and FIG. 7B. FIG. 7A shows a cross-section of the structure prior to a molecular binding event. The depicted structures are wire 700, the glass substrate 702 (n=1.4832), and water 704 (n=1.34) as the dielectric, as is the top superstrate 706 (n=1.348). Layer 708 (before, n=1.348, h=40 nm; after, n=1.388, h=5, 10, or 20 nm) is the protein layer that was added across the entire width of the unit cell for convenience, even though its effect is most important adjacent to the wire. Prior to the binding event, the protein is in an elongated state, about 40 nm in thickness, as shown in FIG. 7A. After the binding event, the protein folds and is only about 5 nm thick, as shown in FIG. 7B.

Electromagnetic field intensity map of the SP fields along the grating show the SP fields are exceptionally high and localized near the surface of the gold wire. Thus, the SP field is most concentrated in the same area of the device where the most pronounced change in refractive index occurs during target binding, resulting in the extreme sensitivity of SPR detection.

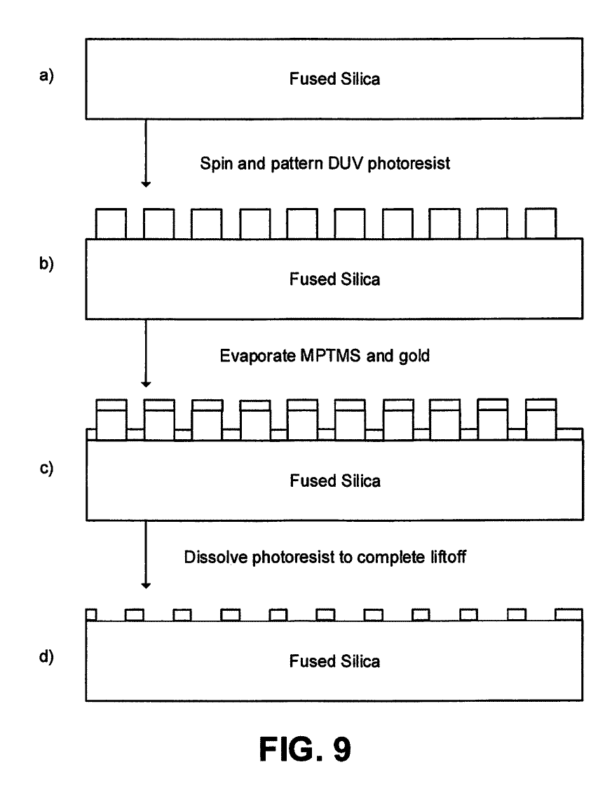

As the protein layer (n=1.388) is compressed, it is displaced by water (n=1.348), changing the transmission, as shown in FIG. 9. The change in transmission of normal-incidence 855-nm light is calculated to be at least 7%, and the greater the thickness of the bound protein layer (for instance, if a larger target molecule is bound), the greater the change in the transmission. Even a 7% change is easily detectable using commercially available optical components.

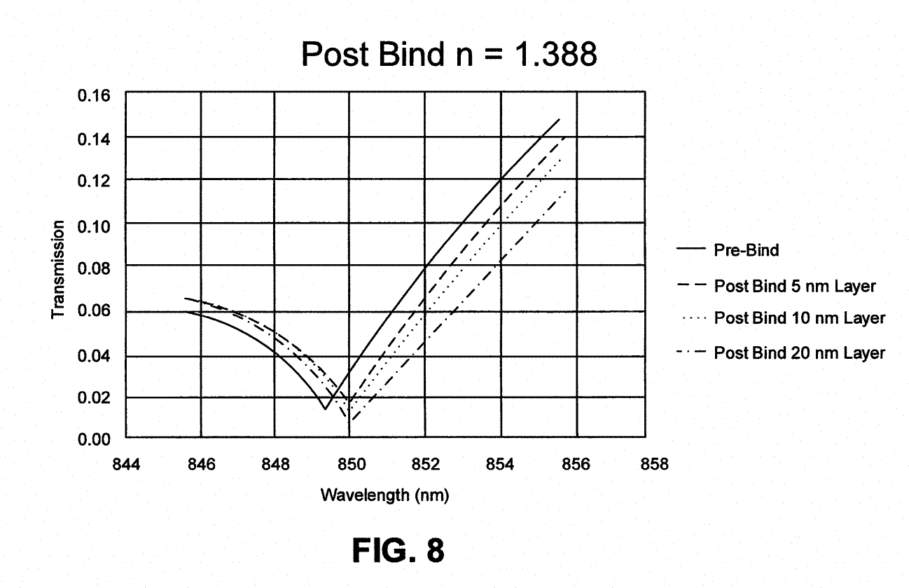

FIG. 8 depicts a graph of transmission of grating as a function of wavelength before and after binding; transmission was measured using the S 2,1 parameter (i.e., 0th-order transmission); index of refraction is n=1.388, and thickness of bound protein layer is 5, 10, or 20 nm.

Taken together, these simulations indicate that a dramatic enhancement in SPR is expected at the surface of protein-bearing gold grating metasurfaces with dimensions which are easily achievable using standard complementary metal-oxide-semiconductor (CMOS) fabrication techniques. To experimentally test these predictions, we have developed a fabrication process for the optimized structure for the plasmonic grating structures onto which the proteins are to be bound. This approach, outlined in FIG. 9, uses a liftoff process to produce a gold film, with a grating pattern of alternating rectangular lines and spaces, on the surface of a fused silica substrate.

First, the back side of the fused silica wafer was sputter-coated with a thin layer of chromium, to enable the exposure tool to detect the transparent substrate. Next, the top side of the wafer was spin-coated with an antireflective coating ARC® DS-K101 (Brewer Science Inc., Rolla, Mo.), and a deep ultraviolet (DUV) negative tone photoresist, UVN®30 (MicroChem Corp., Newton Mass.), which were lithographically patterned. The exposure was performed at 248 nm using a DUV stepper (ASML Holding NV, Veldhoven, Netherlands). Following a post-exposure bake on a hotplate at 95° C. for 90 s, each wafer was developed in AZ®726 MIF (MicroChemicals GmbH, Ulm, Netherlands). Next, a monolayer of (3-mercaptopropyl)trimethoxysilane (MPTMS) (Gelest Inc., Morrisville Pa.) was deposited from the vapor phase, to act as an adhesion promoter for a 50-nm thick layer of gold, which was immediately thereafter evaporated onto the surface. Finally, the wafer was soaked overnight in an organic solvent, MICROPOSIT™ Remover 1165 (MicroChem Corp, Newton Mass.) to dissolve the remaining photoresist, removing with it the excess gold outside the desired patterns. Any remaining DS-K101 was removed by soaking the wafer in AZ®726 MIF.

Preliminary results of this fabrication process at the resist exposure step investigated a range of UV exposure doses. Each square diffraction grating pattern corresponds to a single plasmonic chip. Gratings which are visible from both the top side and back side of the wafer indicate exposure of the resist throughout its entire depth, which is desirable for a successful liftoff process.

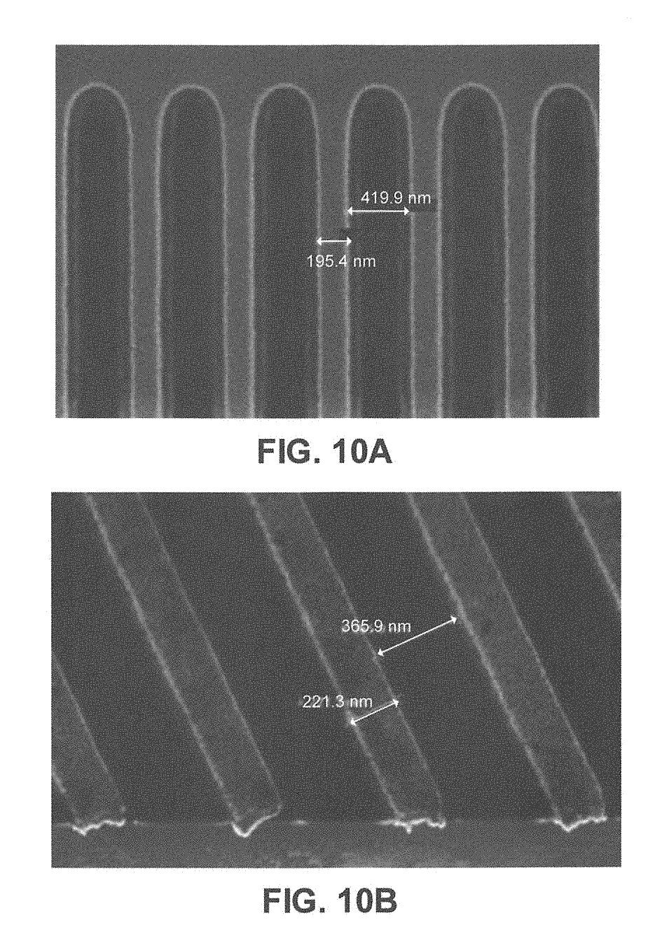

The full fabrication process, including the gold deposition and liftoff, was subsequently performed on this wafer. Several grating structures were studied in greater detail by scanning electron microscopy (SEM), viewed both directly from above (as in FIG. 10A), and in cleaved cross-section (as in FIG. 10B). At 80,000× magnification (FIG. 10A), the edges of the gold wires (light grey) look extremely straight when viewed from above. Furthermore, the observed wire width of 195 nm and spacing of 420 nm are extremely close to the targeted values derived from simulations, of 210 nm and 420 nm, respectively. In cross-section (FIG. 10B), the measurements of the device dimensions are less reliable, because the sample is at an angle relative to the detector. However, this angle clearly confirms both the complete removal of any residual photoresist and antireflective coating from between the gold wires, and the extreme smoothness of the gold wires themselves, both of which are necessary for a properly functioning plasmonic chip.

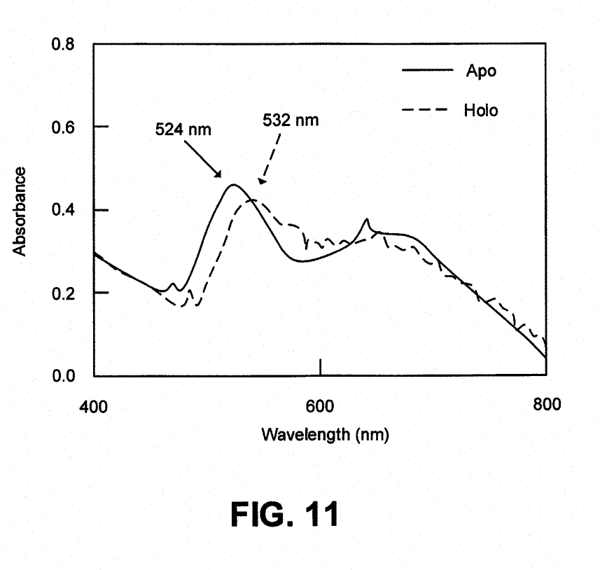

The method was also tested by binding a supercharged protein to a gold nanopartical using a streptavidin-biotin linking strategy and showed, using the change in the nanopartical absorption spectrum. The charge surface electrostatics was more than 1000-fold larger than that observed with small molecules binding to rigid antibodies (see FIG. 11). Given the extra distance imparted by the self-assembled monolayer and the biotin-streptavidin pair, and the electric field screening caused by the charged side chains in streptavidin, this represents a lower limit to the signal enhancement. A shorter distance simplified attachment strategy is believed to increase the enhancement by more orders-of-magnitude. FIG. 11 depicts a gold nanopartical absorption frequency shift upon heme binding to the model protein H4(−28) (SEQ ID NO: 17) upon heme binding. The nanoparticles were coated with a thio-terminated hexanol monolayer doped with biotin thioalkanes, and H4(−28) was expressed as a chimera with streptavidin to bind it to the metal surface.

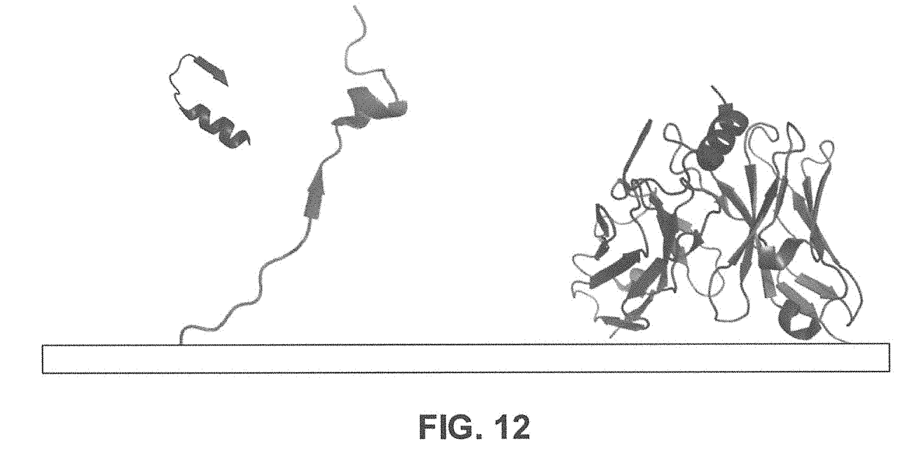

FIG. 12 is an image of a SPR sensor using a supercharged ricin-binding short-chain antibody fragment (scFv). A series of supercharged antibody fragments, termed scFvs for short-chained antibody fragments, that bind and recognize epitopes important for the bio-terro agents Ricin and Botulinum. Starting with either a known Fab monoclonal antibody structure or a homology modeled monoclonal structure derived from a known sequence, the light and heavy-chain hypervariable regions and connect them with a 15-25 residue glycine-rich linker, forming an scFv. Several linker lengths were screened in constructs which have either the heavy chain hypervariable region at the N-terminus and the light chain hypervariable region at the C-terminus or light chain hypervariable region at the N-terminus and the heavy chain hypervariable region at the C-terminus. The screen is for expression, stability and tight epitope binding as detected using isothermal titration calorimetry. The amino acid side chains were identified which are solvent-exposed on the best-performing scFv and mutated a fraction of them such that greater than 15% of the protein residues are negatively charged. Variant sequences, all of which are mutated at a different subset of residues but have similar net charge were created and screened.

The following sequences represent a range of supercharged charge densities for two specific scFv designs.

SEQ ID NO: 1 is a Ricin scFv referred to as 6C2-Neg40-HL. This is a slightly more neutral ricin-binding scFv with heavy chain at the N-terminus. The surface charges have been shuffled in order to change the charge density of the structure.

SEQ ID NO: 2 is a Ricin scFv referred to as 6C2-Neg42-HL. This is the same sequence as the 6C2-Neg40-HL, but with the light chain at the N-terminus, allowing for small differences in binding affinity.

SEQ ID NO: 3 is a Ricin scFv referred to as 6C2-Neg31-HL. This is a more neutral ricin-binding scFv than the other sequences. This will decrease the surface charge density, thereby decreasing the signal, but increasing the binding affinity.

SEQ ID NO: 4 is a Ricin scFv referred to as 6C2-Neg31-LH. This sequence is the same as 6C2-Neg31-HL, but with the light chain at the N-terminus. This switching of domains will have small effects on the charge-charge interactions and binding affinity.

SEQ ID NO: 5 is a Ricin scFv referred to as 6C2-Neg20-HL. This is the least charged ricin-binding scFv with the heavy chain at the N-terminus. This sequence will have the lowest signal, but have the highest affinity.

SEQ ID NO: 6 is a Ricin scFv referred to as 6C2-Neg20-LH. This is also the lowest charged ricin-binding scFv, but with the light chain at the N-terminus. This will shuffle the charges slightly and have small effects on the charge-charge interactions and binding affinity.

SEQ ID NO: 7 is a Ricin scFv referred to as 6C2-Neg42-LH. This is the most charged ricin-binding scFv with the light chain at the N-terminus. Due to its high-charge character it will have the largest signal, but will have the lowest binding affinity out of all the designed proteins.

SEQ ID NO: 8 is a Ricin scFv referred to as 6C2-Neg42-HL. This sequence has the same charge as 6C2-Neg42-LH, but the heavy chain and light chain have been switched, so that the heavy chain is at the N terminus. This allows for subtle differences in charge-charge interactions and binding affinity.

SEQ ID NO: 9 is a Botulinum scFv referred to as CR1-Neg41-HL. This is the most highly charged botulinum-binding scFv with the heavy chain at the N-terminus. Its high charge character will produce the largest signal, but it will consequently have weak binding.

SEQ ID NO: 10 is a Botulinum scFv referred to as CR1-Neg41-LH. This is the same as CR1-Neg41-LH, but with the light chain at the N-terminus. This will have small effects on the binding affinity and charge-charge interaction.

SEQ ID NO: 11 is a Botulinum scFv referred to as CR1-Neg36-HL. This is a less charged version of the botulinum-binding scFv with the heavy chain at the N-terminus. This will have a smaller signal than the higher charged version, but will have a higher binding affinity.

SEQ ID NO: 12 is a Botulinum scFv referred to as CR1-Neg36-LH. This is the same as CR1-Neg36-HL, but with the light chain at the N-terminus. This shuffling of the sequence will subtly change the charge-charge interactions and binding affinity.

SEQ ID NO: 13 is a Botulinum scFv referred to as CR1-Neg30-HL. This is a moderately charged botulinum-binding scFv with the heavy chain at the N-terminus. It is less charged than the previous and will have a lower signal and higher binding affinity.

SEQ ID NO: 14 is a Botulinum scFv referred to as CR1-Neg30-LH. This sequence is the same as CR1-Neg30-HL, but with the light chain at the N-terminus. This switching of domains will have small effects on the charge-charge interactions and the binding affinity.

SEQ ID NO: 15 is a Botulinum scFv referred to as CR1-Neg24-HL. This is the lowest charged botulinum binding scFv with the heavy chain at the N-terminus. It will have the smallest signal, but the highest binding affinity.

SEQ ID NO: 16 is a Botulinum scFv referred to as CR1-Neg24-LH. This is the same sequence as CR1-Neg24-HL, but with the light chain at the N-terminus. The switched domains will have small effects on the signal and binding affinity.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An optical surface plasmon resonance (SPR) biosensor comprising
a light source;
a plasmonic chip with a plurality of intrinsically disordered proteins (IDPs) comprising negatively charged residues, the plurality of IDPs being covalently bond to a plasmonic chip and providing a binding site for binding to a predetermined analyte, wherein the IDPs undergo enzyme-free folding from an extended state to a folded state upon binding to the predetermined analyte;
an optical detector for detecting light from the light source after the light has interacted with the plasmonic chip, the plasmonic chip being disposed between the light source and the optical detector such that the optical detector detects transmitted light.

2. The optical surface plasmon resonance (SPR) biosensor as recited in claim 1, wherein the plurality of IDPs are disposed in a layer that is less than 100 nm thick.

3. The optical surface plasmon resonance (SPR) biosensor as recited in claim 1, wherein the plurality of IDPs are disposed in a layer that is less than 50 nm thick.

4. The optical surface plasmon resonance (SPR) biosensor as recited in claim 1, wherein each IDP consist of residues, and at least 15% of the residues are negatively charged and fewer than 5% of the residues are positively charged.

5. The optical surface plasmon resonance (SPR) biosensor as recited in claim 1, wherein the IDPs have between two-hundred and three-hundred residues.

6. The optical surface plasmon resonance (SPR) biosensor as recited in claim 1, wherein the predetermined analyte is heme and the plurality of IDPs are Histone H4 Protein (non-modified a.a. 1-28) proteins.

7. The optical surface plasmon resonance (SPR) biosensor as recited in claim 1, wherein the plurality of IDPs are short-chain antibody fragments that are antibodies for the predetermined analyte, wherein the short-chain antibody fragments have been modified such that at least 15% of the residues are negatively charged.

8. The optical surface plasmon resonance (SPR) biosensor as recited in claim 7, wherein fewer than 5% of the residues are positively charged.

9. The optical surface plasmon resonance (SPR) biosensor as recited in claim 1, wherein the plurality of IDPs comprises at least one positively charged residue in addition to the negatively charged residues, wherein there are at least three negatively charged residues for each positively charged residue.

10. The optical surface plasmon resonance (SPR) biosensor as recited in claim 1, wherein the plurality of IDPs are characterized by
2.785 [symbol] H [symbol] − [symbol] R [symbol] <1.151
wherein [symbol] H [symbol] is mean hydrophobicity per residue and [symbol] R [symbol] is absolute mean net charge per residue.

11. An optical surface plasmon resonance (SPR) biosensor comprising
a light source
a plasmonic chip with a plurality of intrinsically disordered proteins (IDPs) comprising negatively charged residues, the plurality of IDPs being covalently bond to a plasmonic chip and providing a binding site for binding to a predetermined analyte, wherein the IDPs have between two-hundred and three-hundred residues and undergo enzyme-free folding from an extended state to a folded state upon binding to the predetermined analyte, wherein at least 15% of the residues are negatively charged and fewer than 5% of the residues are positively charged;
an optical detector for detecting light from the light source after the light has interacted with the plasmonic chip.

12. The optical surface plasmon resonance (SPR) biosensor as recited in claim 11, wherein the plurality of IDPs are short-chain antibody fragments that are antibodies for the predetermined analyte, wherein the short-chain antibody fragments have been modified such that at least 15% of the residues are negatively charged.

13. The optical surface plasmon resonance (SPR) biosensor as recited in claim 12, wherein fewer than 5% of the residues are positively charged

14. The optical surface plasmon resonance (SPR) biosensor as recited in claim 11, wherein the plasmonic chip is disposed between the light source and the optical detector such that the optical detector detects transmitted light.

15. The optical surface plasmon resonance (SPR) biosensor as recited in claim 11, wherein the predetermined analyte is heme and the plurality of IDPs are Histone H4 Protein (non-modified a.a. 1-28).

16. The optical surface plasmon resonance (SPR) biosensor as recited in claim 11, wherein the analyte is ricin and the plurality of IDPs are short-chain ricin antibody fragments that have been modified such that at least 15% of the residues are negatively charged and fewer than 5% of the residues are positively charged.

17. The optical surface plasmon resonance (SPR) biosensor as recited in claim 11, wherein the plurality of IDPs are characterized by
2.785 [symbol] H [symbol] − [symbol] R [symbol] <1.151
wherein [symbol] H [symbol] is mean hydrophobicity per residue and [symbol] R [symbol] is absolute mean net charge per residue.

18. A method for detecting an analyte, the method comprising steps of:
exposing a sample to a biosensor configured for use with a surface plasmon resonance (SPR) sensor, the biosensor comprising a plurality of intrinsically disordered proteins (IDPs) comprising negatively charged residues, the plurality of IDPs being covalently bond to a plasmonic chip and providing a binding site for binding to an analyte, wherein the IDPs undergo enzyme-free folding from an extended state to a folded state upon binding to the analyte;
wherein the sample comprises an analyte;
permitting the analyte to bind to at least one IDP in the plurality of IDPs and induce a conformational change in the at least one IDP from a first confirmation to a second confirmation, wherein the plasmonic chip has a first refractive index when the at least one IDP is in the first confirmation and a second refractive index when the at least one IDP is in the second confirmation;
illuminating the plasmonic chip with light;
detecting the second refractive index, thereby detecting the analyte.


SEQUENCE LISTING (17 sequences)

SEQ ID NO: 1 - protein, 249 aa, Artificial Sequence
  Synthetic sequence ((Ricin scFv) 6C2-Neg40-HL)
GDIVETQSPDSLAVSDGQNATIEYQDSKSVSYMHWNEQKPGQPPELLIYLGSNLESGDGANFEGSGEGTDFTLNIHPVEEEDAATYYCQHKREYPPTEGDGTEVEIDGGSGGSGGSGGSVQGGSGGSGGSGGQDPGSELVDPGDSVELSCEASGDTFTSYWINWVKQRPGQGLEWIGNIYPGSGRENYDENFDNEATLEVDTSSDTVYIQESSLTSEDAAEFYCVRWVYGNFDSALDYWGEGTEVTVDS

SEQ ID NO: 2 - protein, 249 aa, Artificial Sequence
  Synthetic sequence (Ricin scFv) 6C2-Neg40-LH
GQDPGSELVDPGDSVELSCEASGDTFTSYWINWVKQRPGQGLEWIGNIYPGSGRENYDENFDNEATLEVDTSSDTVYIQESSLTSEDAAEFYCVRWVYGNFDSALDYWGEGTEVTVDSGGSGGSGGSGGSVQGGSGGSGGSGGDIVETQSPDSLAVSDGQNATIEYRASKSVSYMHWNQQEPGQPPRLLIYLGSNLESGVGANFEGSGSGTDFTLNIHPVEEEDAATYYCQHKREYPPTFGDGTKVEIK

SEQ ID NO: 3 - protein, 249 aa, Artificial Sequence
  Synthetic Sequence (Ricin scFv) 6C2-Neg31-HL
GDIVLTQSPDSLAVSLGQEATISYEASKSVSYMHWNEQEPGEPPELLIYLGSDLESGVGAEFSGSGSGTNFTLDIHPVEQQDAATYYCQHKREYPPTEGQGTEVEIQGGSGGSGGSGGSVQGGSGGSGGSGGQQPGSELVEPGASVELSCEASGYTFTSYWINWVEQEPGQGLEWIGNIYPGSGRTNYDENFQNQATLTVDTSSSTVYIEVSSLTSEDAAVFYCVEWVYGNFDSALDYWGQGTSVTVSE

SEQ ID NO: 4 - protein, 249 aa, Artificial Sequence
  Synthetic Sequence (Ricin scFv) 6C2-Neg31-LH
GQQPGSELVEPGASVELSCEASGYTFTSYWINWVEQEPGQGLEWIGNIYPGSGRTNYDENFQNQATLTVDTSSSTVYIEVSSLTSEDAAVFYCVEWVYGNFDSALDYWGQGTSVTVSEGGSGGSGGSGGSVQGGSGGSGGSGGDIVLTQSPDSLAVSLGQEATISYEASKSVSYMHWNEQEPGEPPELLIYLGSDLESGVGAEFSGSGSGTNFTLDIHPVEQQDAATYYCQHKREYPPTEGQGTEVEIQ

SEQ ID NO: 5 - protein, 249 aa, Artificial Sequence
  Synthetic Sequence (Ricin scFv) 6C2-Neg20-HL
GDIVETQSPDSLAVSDGQNATIEYRASKSVSYMHWNQQEPGQPPRLLIYLGSNLESGVGANFEGSGSGTDFTLNIHPVEEEDAATYYCQHKREYPPTFGDGTKVEIKGGSGGSGGSGGSVQGGSGGSGGSGGQQPGSELVRPGDSVELSCKASGDTFTSYWINWVKQRPGEGLEWIGNIYPGSGRTNYDEKFDNKATLEVDTSSDTVYIQESSLTSEDAAVFYCVRWVYGNFDSALDYWGEGTSVTVDS

SEQ ID NO: 6 - protein, 249 aa, Artificial Sequence
  Synthetic Sequence (Ricin scFv) 6C2-Neg20-LH
GQQPGSELVRPGDSVELSCKASGDTFTSYWINWVKQRPGEGLEWIGNIYPGSGRTNYDEKFDNKATLEVDTSSDTVYIQESSLTSEDAAVFYCVRWVYGNFDSALDYWGEGTSVTVDSGGSGGSGGSGGSVQGGSGGSGGSGGDIVETQSPDSLAVSDGQNATIEYRASKSVSYMHWNQQEPGQPPRLLIYLGSNLESGVGANFEGSGSGTDFTLNIHPVEEEDAATYYCQHKREYPPTFGDGTKVEIK

SEQ ID NO: 7 - protein, 249 aa, Artificial Sequence
  Synthetic Sequence (Ricin scFv) 6C2-Neg42-LH
GEEPGSELVEPGASVELSCEASGYTFTSYWINWVEQEPGEGLEWIGNIYPGSGRTNYDEEFEDEATLTVDTSSSTVYIEVSSLTSEDAAVFYCVEWVYGNFDSALDYWGEGTSVTVSSGGSNSGGSGGSGGSGGSGGSGGSGGDIVLTQSPASLAVSLGEEATISYEASKSVSYMHWNEQEPGEPPELLIYLGSDLESGVGAEFSGSGSGTDFTLDIHPVEEEDAATYYCQHKREYPPTFGEGTEVEIE

SEQ ID NO: 8 - protein, 249 aa, Artificial Sequence
  Synthetic Sequence (Ricin scFv) 6C2-Neg42-HL
GDIVLTQSPASLAVSLGEEATISYEASKSVSYMHWNEQEPGEPPELLIYLGSDLESGVGAEFSGSGSGTDFTLDIHPVEEEDAATYYCQHKREYPPTFGEGTEVEIEGGSNSGGSGGSGGSGGSGGSGGSGGEEPGSELVEPGASVELSCEASGYTFTSYWINWVEQEPGEGLEWIGNIYPGSGRTNYDEEFEDEATLTVDTSSSTVYIEVSSLTSEDAAVFYCVEWVYGNFDSALDYWGEGTSVTVSS

SEQ ID NO: 9 - protein, 263 aa, Artificial Sequence
  Synthetic Sequence CR1 (Botulinum scFv) CR1-Neg41-HL
DLGGSGENLYFQGVELQESGGGEVDPGGSEELSCAADGDTFKYDYMYWVRQAPGDGLEWVATISDGGSYTYYSDSVEGRFETSRDNSKETLELQMNSLDAEDTAIYYCSRYRYDDAMDEWGQGTEVEVGGSGGSGGSGGSVQGGSGGSGGSGGEIVLTQSPDTLSLSPGEDAEISCRASESVDSYGHSFMQWYQQKPGDDPELLIYRASELEPGDPARFSGEGSGTDFTLTIDSLEPEDFAEYYCQQGNEVPFTDGQGTEVEE

SEQ ID NO: 10 - protein, 263 aa, Artificial Sequence
  Synthetic Sequence CR1 (Botulinum scFv) CR1-Neg41-LH
DLGGSGENLYFQGEIVLTQSPDTLSLSPGEDAEISCRASESVDSYGHSFMQWYQQKPGDDPELLIYRASELEPGDPARFSGEGSGTDFTLTIDSLEPEDFAEYYCQQGNEVPFTDGQGTEVEEGGSGGSGGSGGSVQGGSGGSGGSGGVELQESGGGEVDPGGSEELSCAADGDTFKYDYMYWVRQAPGDGLEWVATISDGGSYTYYSDSVEGRFETSRDNSKETLELQMNSLDAEDTAIYYCSRYRYDDAMDEWGQGTEVEV

SEQ ID NO: 11 - protein, 263 aa, Artificial Sequence
  Synthetic Sequence CR1 (Botulinum scFv) CR1-Neg36-HL
DLGGSGENLYFQGDQLQESGGGEEQPGGSENLSCEESGFTFKYDYMYWVRQDPGQGEEWVATISDGGSYTYYSDSVEGRFTTSRDNSKDTLELQMNSLNAEDTAEYYCSRYRYDDAMDEWGQGTEVTVGGSGGSGGSGGSVQGGSGGSGGSGGEIDLEQDPAELSESPGENAEISCRASESVDSYGHSFMQWYQQKPGDDPRLLIYRADNLEPGDPARFSGSGSGTDFTLTISDLEPEDFAVYYCQQGNEVPFTFGQGTDVEI

SEQ ID NO: 12 - protein, 263 aa, Artificial Sequence
  Synthetic Sequence CR1 (Botulinum scFv) CR1-Neg36-LH
DLGGSGENLYFQGEIDLEQDPAELSESPGENAEISCRASESVDSYGHSFMQWYQQKPGDDPRLLIYRADNLEPGDPARFSGSGSGTDFTLTISDLEPEDFAVYYCQQGNEVPFTFGQGTDVEIGGSGGSGGSGGSVQGGSGGSGGSGGDQLQESGGGEEQPGGSENLSCEESGFTFKYDYMYWVRQDPGQGEEWVATISDGGSYTYYSDSVEGRFTTSRDNSKDTLELQMNSLNAEDTAEYYCSRYRYDDAMDEWGQGTEVTV

SEQ ID NO: 13 - protein, 263 aa, Artificial Sequence
  Synthetic Sequence CR1 (Botulinum scFv) CR1-Neg30-HL
DLGGSGENLYFQGDQLQESGGGLEQPGGSENLSCAESGDTFKYDYMYWVRQDPGQGLEWVATISDGGSYTYYSDSVEGRFTTSRDNSKDTLYLQMNSLNAEDTAEYYCSRYRYDDAMDYWGQGTEVTEGGSGGSGGSGGSVQGGSGGSGGSGGEIDLTQDPAELSESPGENAEISCRASESVDSYGHSFMQWYQQKPGDAPNLLIYRASNLEPGIPARFSGEGSGTDFTLTISDLEPEDFAVYYCQQGNEVPFTFGQGTNVEI

SEQ ID NO: 14 - protein, 263 aa, Artificial Sequence
  Synthetic Sequence CR1 (Botulinum scFv) CR1-Neg30-LH
DLGGSGENLYFQGEIDLTQDPAELSESPGENAEISCRASESVDSYGHSFMQWYQQKPGDAPNLLIYRASNLEPGIPARFSGEGSGTDFTLTISDLEPEDFAVYYCQQGNEVPFTFGQGTNVEIGGSGGSGGSGGSVQGGSGGSGGSGGDQLQESGGGLEQPGGSENLSCAESGDTFKYDYMYWVRQDPGQGLEWVATISDGGSYTYYSDSVEGRFTTSRDNSKDTLYLQMNSLNAEDTAEYYCSRYRYDDAMDYWGQGTEVTE

SEQ ID NO: 15 - protein, 263 aa, Artificial Sequence
  Synthetic Sequence CR1 (Botulinum scFv) CR1-Neg24-HL
DLGGSGENLYFQGVELQESGGGEVDPGGSLRLSCAADGFTFKYDYMYWVRQAPGKGEEWVATISDGGSYTYYSDSVEGRFETSRDNSKNTLELQMNSLRAEDTAEYYCSRYRYDDAMDYWGQGTLVTVGGSGGSGGSGGSVQGGSGGSGGSGGEIVLTQSPDTLSDSPGERATISCRASESVDSYGHSFMQWYQQKPGQDPELLIYRASNLEPGDPARFSGEGSGTDFTLTIDSLEPEDFAEYYCQQGNEVPFTDGQGTKVEI

SEQ ID NO: 16 - protein, 263 aa, Artificial Sequence
  Synthetic Sequence CR1 (Botulinum scFv) CR1-Neg24-LH
DLGGSGENLYFQGEIVLTQSPDTLSDSPGERATISCRASESVDSYGHSFMQWYQQKPGQDPELLIYRASNLEPGDPARFSGEGSGTDFTLTIDSLEPEDFAEYYCQQGNEVPFTDGQGTKVEIGGSGGSGGSGGSVQGGSGGSGGSGGVELQESGGGEVDPGGSLRLSCAADGFTFKYDYMYWVRQAPGKGEEWVATISDGGSYTYYSDSVEGRFETSRDNSKNTLELQMNSLRAEDTAEYYCSRYRYDDAMDYWGQGTLVTV

SEQ ID NO: 17 - protein, 131 aa, Artificial Sequence
  Synthetic Sequence H4(-28)
GEIWQQHEEALQQFEEALQQFEELQQLGGSNSGSGEIWQQHEEALQQFEEALQQFEELQQLGGGGSSTGSGEIWQQHEEALQQFEEALQQFEELQQLGGSSGGSGEIWQQHEEALQQFEEALQQFEELQQL